(19) United States
(12) Patent Application Publication
(10) Pub. No.: US 2026/0216672 A1
(43) Pub. Date: Jul. 30, 2026

(54) CURING DEVICE FOR CURING CAPSULES

(71) Applicant: MICROCAPS AG, Schlieren (CH)

(72) Inventors: Enea BAUMANN, Zürich (CH); Christian BURKARD, Aarau (CH); David SCHAFFNER, Rheinfelden (CH)

(21) Appl. No.: 19/150,711

(22) PCT Filed: Jan. 15, 2024
(86) PCT No.: PCT/EP2024/050784

§ 371 Date: Jul. 24, 2025

(30) Foreign Application Priority Data
Jan. 26, 2023 (CH) CH000065/2023

(51) Int. Cl. B01F 23/43; A23P 10/30; A61K 9/50; B01F 23/41; B01F 27/112; B01F 27/192; B01F 27/90; B01F 35/21; B01F 35/71; B01F 35/90; B01F 35/92

(52) U.S. Cl. CPC B01F 23/43; A61K 9/5089; B01F 23/4105; B01F 27/112; B01F 27/192; B01F 27/90; B01F 35/2115; B01F 35/71805; B01F 35/92; A23P 10/30; B01F 2035/99; B01F 2215/0422

(57) ABSTRACT

Disclosed herein is a curing device (1) for generating and curing capsules, which comprises a tubular column (2), a bottom portion (5), a head portion (8) and a radial stirring device (10) being arranged inside the tubular column (2) and inside a stirring zone (4) of the tubular column (2). Furthermore, a capsule production unit and a method for generating and curing capsules is disclosed.

### FIELD OF DISCLOSURE

The present invention lies in the field of capsule production technology, in particular capsules having been made by melting a matrix material, generating a dispersion of the molten matrix material to form droplets of the molten matrix material and solidify the droplets by cooling to form capsules. The present invention relates to a curing device for curing capsules, a capsule production unit with such a curing device and a method of generating and curing capsules.

### BACKGROUND, PRIOR ART

Encapsulation of compounds of interest has gained profound significance in many different fields. Besides the encapsulation of active pharmaceutical ingredients in pharmaceutical sciences, the encapsulation of fragrances and flavors in food technology and cosmetic substances in cosmetic products has found increased application. Encapsulation offers many advantages which can be exploited depending on the desired application. For example, ingredients, such as fragrances or flavors can be better conserved, or the encapsulated substances can be released only in a specific environment or under specific conditions. This is beneficial for pharmaceutical applications, in which an encapsulated active pharmaceutical ingredient shall only be liberated in the acidic environment of the stomach. This can be achieved by an acid labile, but otherwise stable capsule body or shell. In food technology, it may for example be desirable to encapsulate flavors in an oil or wax matrix material which only releases the flavors upon heating the capsules to a specific temperature. An application example may be the encapsulation of smoke flavor in a fat capsule for a meat substitute product. Upon cooking the product, the fat melts and releases the encapsulated flavor. The above mentioned applications are only an illustrative selection for the various different applications of encapsulated substances.

In general, two different capsule types are known, namely massive capsules which are made of a matrix material and contain optionally additionally one or more compounds of interest, and shell-core capsules. In contrast to massive capsules, shell-core capsules contain a shell of a matrix material, which encapsulate an often liquid core. A prominent example are oil core capsule with polysaccharide shells. These capsules contain a liquid oil core, which may also include a lipophilic compound of interest, and a solid polysaccharide shell surrounding the oil core. Most prominently, calcium alginate shells are used. However, it is also possible to use other materials.

For preparing capsules, in particular massive capsules, emulsification is beneficial, because it allows for a relative accurate size control of the capsules and may at least to some extent and depending on the operational setup even allow high-throughputs. A large portion of known massive capsules can be prepared by melting a matrix material, thereby transforming it from a solid to a liquid state (or at least from a viscous to a less viscous state) and mix it with a continuous phase, which is not miscible with the molten matrix material, to provide a dispersion of the molten matrix material (the dispersed phase) in the continuous phase. The molten matrix material is then present as liquid droplets in the continuous phase. If the dispersion is then cooled below the melting temperature of the matrix material, the droplets solidify and form capsules. This process is referred to as generating and curing capsules. If the molten matrix material is prior to emulsification mixed with a suitable compound of interest, in particular a compound of interest which is soluble in the molten matrix material, then the substance is also at least partially incorporated into the capsules. A suitable emulsification device which can be used is for example disclosed in WO 2021 037 999 A2, which is included herein by reference in its entirety. For producing massive capsules, a preferred system is to use a wax, a fat or oil as matrix material which can generally be referred to as an oil phase, and an aqueous phase, e.g. water, as the continuous phase. The wax, fat or oil can be molten and then be dispersed in an aqueous continuous phase. Upon cooling the dispersion, for example in a stirred batch reactor, the droplets of the molten wax or oil solidify, thereby generating cured capsules.

A common problem during preparation of such massive capsules being produced by melting-dispersing-cooling as described above is that the droplets of molten matrix material and also the partially formed and partially cured capsules start to agglomerate, or adhere to reactor surfaces, such as stirrers, reactor walls, tubing, filter and the like. While there are at least to some extent ways to minimize these problems, it often requires cumbersome procedures and often leads to extended production times, which is disadvantageous. Shortening the production time however, often leads to the occurrence of agglomeration or adhesion to surfaces and therefore to a decrease in capsule quality.

### SUMMARY OF DISCLOSURE

It is therefore the general object of the invention to advance the state of the art in the field of capsule production, in particular for producing massive capsules as outlined above. Preferably, the disadvantages of the prior art mentioned above are at least partially or fully overcome. In some advantageous embodiments devices and methods are provided which decrease or even completely avoid capsule agglomeration or adhesion to surfaces. In advantageous embodiments, devices and methods are provided, which allow to achieve a high capsule quality, e.g. high size control, constant capsule quality and concomitantly shorten the production time. In further advantageous embodiments, devices and methods are provided, which facilitate the production of capsules and particularly allow to increase automation of the production process.

The general object is achieved by the subject-matter of the independent claims. Further advantageous embodiments follow from the dependent claims and the overall disclosure.

In a first aspect, the general object is achieved by a curing device for generating and curing capsules. Particularly, the invention relates to massive capsules as described above. Typically, these capsules are produced by melting-dispersing-cooling. In preferred embodiments, the capsules comprise a solid matrix, i.e. a matrix being solid at room temperature (20° C.) and normal pressure (1 atm.). The curing device according to the invention comprises a tubular column which has a longitudinal axis. The longitudinal axis extends along an axial direction of the tubular column and thus, as outlined below, typically extends through and between a head portion and a bottom portion of the curing device. The tubular column defines, respectively comprises, along the longitudinal axis a pre-curing zone of the tubular column and an adjacent thereto stirring zone of the tubular column. The curing device further comprises a head portion and a bottom portion. The tubular column is arranged between the head portion and the bottom portion. The bottom portion comprises a first fluid inlet unit and a therefrom separated second fluid inlet unit. The first fluid inlet unit is configured for introducing a dispersion, such as droplets of a molten matrix material in a first continuous phase, into the tubular column, and particularly into the pre-curing zone and the second fluid inlet unit is configured for introducing a second continuous phase into the tubular column, in particular into the pre-curing zone. The pre-curing zone extends between the bottom portion of the curing device and the stirring zone of the tubular column, in particular directly between the bottom portion of the curing device and the stirring zone. The head portion comprises a fluid outlet for removing the generated and cured capsules from the tubular column and/or the curing device. The curing device further comprises a radial stirring device being arranged inside the tubular column and inside the stirring zone defined by the tubular column. The radial stirring device is configured such that it can provide a radial mixing of the dispersed phase and the continuous phase within the tubular column and in particular within the stirring zone of the tubular column. The radial stirring device particularly extends along the longitudinal axis of the curing device from the head portion only into the stirring zone. Therefore, the stirring device does particularly not extend into the pre-curing zone of the tubular column. The pre-curing zone is therefore particularly devoid of the stirring device.

It is understood that the stirring zone and the pre-curing zone do not have to be separated from each other by a physical element, such as a wall or the like. It is preferred that the pre-curing zone transitions into the stirring zone. However, they particularly differ from each other in that the radial stirring device extends only into the stirring zone, but not into the pre-curing zone. Thus, the pre-curing zone is in particular free of the radial stirring device, and preferably of any other stirring device. The dispersion with the droplets of the molten matrix material (and optionally also partially cured and partially generated capsules) and the continuous phase from the second fluid inlet unit are both introduced into the pre-curing zone and are contacted with each other therein. Since the continuous phase being introduced via second fluid inlet unit has a temperature being below the melting temperature of the matrix material of the droplets, the droplets start to cure (e.g. to harden and/or solidify) and generate droplets in the pre-curing zone. In the initial state, the partially cured and partially generated capsules have a large tendency to agglomerate and adhere to surfaces. Furthermore, they are in this state very labile. By providing a pre-curing zone without a stirring device, the droplets can solidify and cure to a certain extend beyond a critical degree without being damaged by the stirring device and without adhering to surface of the stirring device, such as the stirring elements. This may be referred to as “pre-curing”. They can then leave the pre-curing zone having cured beyond a critical level into the stirring zone in which the capsules can cure further before they can be expelled from the curing device via the fluid outlet of the head portion.

It is generally understood herein that the term “comprising” is interpreted as meaning that it includes those features following this term, but that it does not exclude the presence of other features, as long as they do not render the claim unworkable. On the other hand, if the wording “consist of” is used, then no further features are present in the corresponding apart from the ones following said wording.

In certain embodiments, the tubular column consists only of the pre-curing zone and the stirring zone and has thus no additional zone.

It is understood that the longitudinal axis of the tubular column is arranged in the center of the tubular column. In particular, the longitudinal axis has in any radial direction the same distance to the column walls of the tubular column. The tubular column may in general comprise one or more column walls which define a fluid pathway for the curing and generating capsules from the first and second fluid inlet unit to the fluid outlet.

The tubular column is in some embodiments cylindrical. In particular, the tubular column may define a cylindrical chamber. It is understood that the tubular column has a head opening and a bottom opening. Typically, the bottom portion of the curing device is attached to the bottom opening of the tubular column and the head portion of the curing device is attached to the head opening of the tubular column. It is understood that the terms “top” and “bottom” do not necessarily mean that the top portion is in the 3D space in the vertical direction (i.e. against the gravitational force vector) necessarily above the bottom portion. While this is however possible, it is also possible to turn the device by 90° or 180° C., for example if capsules are generated which due to the density of their oily matrix material are heavier than the continuous phase. Furthermore, it may be possible to use the device in any possible orientation with respect to the gravitational vector, e.g. in a horizontal manner, i.e. 90° to the gravitational force vector. The tubular column has column walls, which define the column chamber, i.e. the chamber into which the continuous phase from the second fluid inlet unit and the dispersion from the first fluid inlet unit are provided. In some embodiments, the tubular column may essentially be rotationally symmetric with respect to its longitudinal axis.

In some embodiments, the tubular column, the radial stirring device, the head portion and/or the bottom portion comprise, or consist of, metal, in particular steel, glass and/or of a polymer material.

In some embodiments, the length, i.e. the extension of the tubular column in the axial direction, to width, respectively the diameter, of the tubular column is at least 3:1. Particularly, the length to width of the tubular column is between 3:1 and 50:3, in particular between 5:1 and 10:1. Thus, the flow behavior within the tubular column typically resembles a pipe flow. In particular, the length of the tubular column may in some embodiments be between 15 cm and 60 cm, in particular between 30 cm and 45 cm. In some embodiments, the width, respectively the diameter of the tubular column is between 20 mm and 100 mm, in particular between 30 mm and 80 mm, in particular between 40 mm and 60 mm.

In some embodiments, the radial stirring device is configured such that a radial vortex is generated when stirring the dispersed phase and the continuous phase.

In some embodiments, the radial stirring device comprises, or consists of a stirrer rod and one or more stirring elements. The stirring elements may be connected to the stirrers rod and preferably extend at least partially radially from the stirrer rod.

In some embodiments the radial stirring device comprises at least two stirrer elements. Preferably, one stirrer element is arranged closer to the fluid outlet of the head portion than the other one. In particular embodiments, one stirrer element has in the longitudinal direction a distance to the fluid outlet of the head portion of between 10 cm to 50 cm, in particular 20 cm to 30 cm. Such a close arrangement to the fluid outlet ensures proper mixing at the fluid outlet to prevent particle agglomeration and clogging of the outlet.

In some embodiments, the first fluid inlet unit comprises a first inlet tube.

In some embodiments, the second fluid inlet unit comprises a plurality of second inlets, which are arranged around the first inlet tube. Thereby, the continuous phase from the second fluid inlet unit (which may be referred to as a second continuous phase), does not create undesired turbulences and does not damage the droplets, respectively the partially cured and partially generated capsules. In particular embodiments, the second inlets may be arranged on a circle surrounding the first inlet tube. The second inlets may preferably be spaced apart from the first inlet tube. The second inlets and the first inlet tube may typically be arranged such that fluid expelled therefrom flows essentially in the same direction, e.g. in the axial direction.

In some embodiments, the first inlet tube comprises a heating element, such as a heating coil, being configured to provide thermal energy to the dispersed phase flowing therethrough. Such a heating coil prevents curing of the droplets while passing through the first inlet tube and therefore avoids clogging.

In some embodiments, the second fluid inlet unit comprises at least 4, in particular at least 6, more particular at least 8, more particular at least 10, second inlets. In some embodiments, the second fluid inlet unit comprises 4 to 20 second inlets, in particular 6 to 20 second inlets, more particular 8 to 16 second inlets, more particular 10 to 14 second inlets.

It is understood that the term “second inlet” does not necessarily mean that there must be corresponding other first inlets, but indicates that these second inlets are part of the second fluid inlet unit.

In some embodiments, the second fluid inlet unit may comprise a second inlet tube and a flow channel. The flow channel may provide a fluidic connection between the second inlets and the second inlet tube. Typically the second inlet tube is configured for being connected to a continuous phase reservoir.

In some embodiments, the second inlets are configured such that they can generate a vortex of the continuous phase upon introduction into the tubular column, and particularly into the pre-curing zone. The vortex extends along and around the longitudinal axis of the tubular column and transports the droplets and partially cured and partially generated capsules towards the stirring zone. Furthermore, the vortex allows for efficiently mixing the droplets and partially cured and partially generated capsules with the continuous phase from the second fluid inlet unit and also with the first continuous phase. Since the second continuous phase has typically a lower temperature than the first continuous phase, curing of the capsules is improved and curing is achieved in a mild manner, avoiding the contact with a stirring device in the pre-curing zone. This enables that the forming capsules are sufficiently stable and solidified once they reach the stirring zone and do not agglomerate and/or adhere to the radial stirring device.

In some embodiments, each second inlet may form a curved flow path.

In some embodiments, each second inlet of the second fluid inlet unit defines an inlet axis. The corresponding second inlet extends along its inlet axis and/or the inlet axis is defined by the direction along which the continuous phase is expelled from the corresponding second inlet. Each inlet axis is angled towards a horizontal plane in an angle of below 90°, in particular of 50° to 80°, more particular of 55° to 65°. The horizontal plane is perpendicular to the longitudinal axis of the tubular column and thus extends in the radial direction of the tubular column.

In some embodiments, a length of the pre-mixing zone, i.e. its extension along the longitudinal axis of the tubular column, is at least 20%, in particular at least 30%, more particular at least 40%, more particular at least 50%, of a total length of the tubular column. The total length of the tubular column refers to the total extension of the tubular column along the longitudinal axis. In some embodiments, the length of the pre-mixing zone is between 20% to 70%, in particular between 30% to 65%, more particular between 40% to 60%, more particular between 50% to 60%, of a total length of the tubular column. Such a ratio allows the droplets and or generating capsules to cure beyond a critical level and therefore prevents efficiently that the capsules are damaged by or adhere to the radial stirring device. Furthermore, agglomeration is avoided.

In some embodiments, the ratio of the length of the pre-curing zone and the diameter of the pre-curing zone is between 4:1 and 2:1.

In some embodiments, the length of the pre-curing zone is between 100 mm to 200 mm, in particular between 130 mm to 170 mm, more particular between 150 mm to 166 mm.

In some embodiments, the radial stirring device is a paddle stirrer, a blade stirrer or an anchor stirrer.

In some embodiments, the curing device further comprises a temperature sensor unit, which is configured for measuring the temperature of the continuous phase inside the tubular column, and in particular inside the stirring zone. The temperature sensor may preferably be configured for measuring the mixing temperature between the dispersion of droplets including the first continuous phase and the second continuous phase. In certain embodiments, the head portion comprises the temperature sensor unit. The temperature sensor unit may for example comprise a sensor opening and a sensor being inserted into the curing device.

In some general embodiments, the curing device comprises a control unit. The control unit may for example comprise a circuit, particularly a microprocessor. In addition, the curing device may comprise a display and/or input means being configured for providing commands to the control unit, for example a keyboard or a touch screen.

The temperature sensor unit may in certain embodiments be configured for transmitting the measured temperature to the control unit. It may further be possible that the control unit is configured for receiving the measured temperature of the temperature sensor unit and for comparing it with a predefined target temperature. If the control unit determines that the measured temperature exceeds the target temperature, it may be configured to adjust the residence time of the forming capsules within the tubular column, e.g. extent the residence time by decreasing the flow rate with which the continuous phase and/or the dispersion is introduced into the tubular column. Alternatively or additionally, the control unit may be configured to lower the temperature of the continuous phase being introduced into the tubular column via the second fluid inlet unit. This may for example be achieved by adjusting and controlling corresponding cooling elements, which may for example be part of a continuous phase reservoir.

In some embodiments, the fluid outlet of the head portion defines an outlet axis being angled to the longitudinal axis in an angle of 10° to 90°, in particular of 20° to 60°, more particular of 30° to 50°. Such an angle allows to remove the generated and cured capsules without significantly changing the direction of flow and therefore avoid damaging the capsules.

In some embodiments, the head portion comprises an inclined surface which is configured such that generated and cured capsules are guided towards the fluid outlet of the head portion. The inclined surface may be angled towards the horizontal plane being perpendicular to the longitudinal axis of the tubular column in an angle of between >0° and <90°, e.g. between 30° and 75°, e.g. between 50° and 65°. This prevents capsule adhesion to surfaces of the curing device and capsule agglomeration and clogging.

In some embodiments, the head portion further comprises a drive unit, such as a motor, which is configured for rotating the radial stirring device. The drive unit may preferably be a stepper motor. In some embodiments, the drive unit may be controlled by the control unit of the curing device. For example, the control unit may be configured of adjusting the rotation speed and/or rotation direction. In preferred embodiments, the control unit is configured for changing the rotational direction, e.g. between clockwise and counter clockwise rotation, at predefined points in time and/or for predefined time intervals. This allows to break a Taylor-Couette flow generating due to the radial stirring, which in turn avoid agglomeration of the capsules.

In a second aspect, the general object is achieved by a capsule production unit. The capsule production unit comprises a curing device according to any of the embodiments described herein, in particular with respect to the first aspect of the invention. Furthermore, the capsule production unit comprises an emulsification device, which is configured for generating a dispersion of droplets in a continuous phase. The emulsification device is in fluidic connection with the first fluid inlet unit of the curing device.

The emulsification device may typically comprise a first chamber and a second chamber being separated by a membrane having one or more channels, in particular micro-channels. In certain embodiments, the emulsification device is a device as described in WO 2021 037 999 A2 of the applicant, in particular in the independent claim 1 and the dependent claims as well as the figures. In preferred embodiments, the device comprises a first inlet for supplying a first phase (e.g. the molten matrix material), which opens into a first chamber, a second inlet for supplying a second phase (e.g. the first continuous phase), opening into a second chamber and a dispersion outlet for collecting the formed dispersion of droplets in the second phase, i.e. in the first continuous phase (e.g. water or an alcohol, such as polyvinyl alcohol). Furthermore, the device may comprise a membrane, which separates the first chamber and the second chamber and which comprises a first side facing the first chamber and a second side facing the second chamber. The membrane may comprise multiple channels extending from the first side to the second side, providing a fluidic connection between the first chamber and the second chamber. Each channel comprises a channel inlet arranged on the first side and a channel outlet arranged on the second side. The first chamber may preferably be configured such that a flow rate of the first phase through all of the individual channels is essentially equal. In some embodiments, the first and second continuous phase may have different temperatures. For example the temperature of the first continuous phase may be higher than the temperature of the second continuous phase. In particular the first continuous phase may be selected such that the molten matrix material remains in a molten and/or liquid state when being in contact with the first continuous phase. The first phase may for example be a molten matrix material, in particular an oil phase, and the second phase (the continuous phase) may be an aqueous phase, such as water. It is clear to the skilled person that the term “oil phase” refers to the lipophilic properties of such a phase, i.e. the oil phase may for example be a wax, an oil or a fat. Accordingly, the term “aqueous phase” refers to the hydrophilic properties and may thus for example be water, an alcohol and/or DMSO. The oil phase and the aqueous phase are typically generally not miscible with each other, in particular at 20° C. and 1 atm. In preferred embodiments, the dispersion outlet of the emulsification device is fluidic connected, in particular directly fluidic connected, to the first fluid inlet unit of the curing device.

In certain embodiments, the dispersion outlet of the emulsification device may essentially be arranged on the central longitudinal axis and/or the axis being perpendicular to the membrane and intersecting the center of the membrane. Preferably, the second chamber is tapered towards the dispersion outlet. For example, at least parts of the second chamber may be arch- or cone-shaped towards the dispersion outlet. These embodiments ensure that no droplets are entrapped and all are directly collectable via the dispersion outlet.

In some embodiments, the capsule production unit additionally comprises a continuous phase reservoir being in fluidic communication with the second fluid inlet unit of the curing device. The continuous phase reservoir can in particular be configured to provide a second continuous phase. As understood herein, the continuous phase supplied to the emulsification device, e.g. to the second inlet of the emulsification device, and which is used to provide the dispersion of droplets in this continuous phase in the emulsification device, may be termed as “first continuous phase”. The continuous phase being provided into the tubular column of the curing device via the second fluid inlet unit of the curing device may be termed as “second continuous phase”. The first and second continuous phase may be the same, e.g. both be water or an alcohol, such as polyvinyl alcohol, or may be different from each other. However, typically, the first and second continuous phase are miscible with each other at room temperature (20° C.) and normal pressure (1 atm.). The continuous phase reservoir may comprise cooling elements which are configured to cool the continuous phase before it is being provided to the second fluid inlet unit of the curing device. In particular embodiments, the cooling elements may be controlled by the control unit of the curing device.

In some embodiments, the capsule production unit further comprises a processing unit, in particular a processing unit for preparing a molten matrix material, in particular a molten oil phase. The processing unit comprises a melting unit and a dosing unit. The melting unit defines, respectively comprises, a melting compartment. Furthermore, the melting unit comprises a heating unit which is configured for heating a matrix material within the melting compartment. The dosing unit is configured for providing a molten matrix material, in particular a molten oil phase, from the melting unit to the emulsification device, in particular to the first inlet of the emulsification device opening into the first chamber. The processing unit can preferably be used as follows: A solid or at least viscous matrix material is provided to the melting compartment. Then, the heating unit heats the matrix material above its melting temperature, such that it is in a liquid state or in has a reduced viscosity. Then, the dosing unit can deliver the molten matrix material to the emulsification device, in which it forms the droplets of a dispersion of droplets in a (first) continuous phase.

In some embodiments, the dosing unit may comprise a pump, e.g. a syringe pump.

In some embodiments, the melting unit further comprises a matrix material inlet, for example a funnel shaped inlet, which is configured for introducing matrix material, in particular an oil phase, into the melting compartment of the melting unit.

In some embodiments, the processing unit further comprises a valve, such as one or more 2-way valves or a 3-way valve. If the valve comprises two or more 2-way valves the valve may be considered as a valve unit. The valve is fluidic connected to the melting unit, to the dosing unit and also to the emulsification device. Typically, the valve is directly fluidic connected to the melting unit, to the dosing unit and also to the emulsification device. In typical embodiments, the valve is arranged between all of the melting unit, the dosing unit and the emulsification device. The valve allows for an efficient process management, since the matrix material can be molten in the melting unit and the valve provides an exclusive fluidic connection between the melting unit and the dosing unit. The dosing unit can then apply a negative pressure to guide the molten matrix material into the dosing unit. Then the valve is switched such that it provides an exclusive fluidic connection between the dosing unit and the emulsification device. Then the dosing unit can apply a positive pressure and deliver the molten matrix material, in particular the molten oil phase, from the dosing unit to the emulsification device. In the meantime, it is possible to melt further additional matrix material in the melting unit.

In some embodiments, the dosing unit is therefore configured for establishing a negative pressure to transport the molten molten matrix material, in particular the molten oil phase, from the melting unit into the dosing unit via the valve and/or the dosing unit is configured for establishing a positive pressure to transport the molten matrix material, in particular the molten oil phase, from the dosing unit to the emulsification device via the valve.

In some embodiments, the valve is controlled by the control unit of the curing device.

In some embodiments, the melting unit further comprises a stirrer. The stirrer is typically configured for stirring the matrix material in the melting compartment. For example, the stirrer may be an anchor stirrer or a blade stirrer. The stirrer may in some embodiments also be configured for uniformly distributing the heat of the first continuous phase and the second continuous phase.

In some embodiments, the melting unit further comprises an emulsifier unit, such as macerator unit. The emulsifier unit may for example be a rotor-stator. The emulsifier unit is typically arranged at least partially inside the melting compartment. Preferably, the emulsifier unit is configured for emulsifying a compound of interest with the matrix material. Furthermore, the emulsifier unit may be configured to shear, break and/or comminute the matrix material within the melting compartment. Typically the emulsifier unit further comprise a drive unit. The emulsifier unit enables to rapidly provide a homogenous molten matrix material.

In some embodiments, the dosing unit further comprises a dosing unit heater which is configured for heating the dosing unit, respectively for heating the molten matrix material, e.g. the molten oil phase, within the dosing unit. Such a heater prevents that the molten matrix material cools within the dosing unit and therefore solidifies or becomes heterogeneous.

In some embodiments, the distance the dispersion travels between the emulsification device until it reaches the pre-curing zone is between 10 mm to 500 mm, in particular between 270 mm an 340 mm.

The general object is in a third aspect achieved by a method for generating and curing capsules. The method comprises the steps:
a. Providing a dispersion of droplets which comprise a molten matrix material, in a first continuous phase. The molten matrix material may preferably be a molten oil phase and the first continuous phase may preferably be an aqueous phase.
b. Introducing the dispersion into a pre-curing zone of a tubular column of a curing device. Preferably, the dispersion is introduced into the pre-curing zone via a first fluid inlet unit of a bottom portion of the curing device.
c. Introducing a second continuous phase into the pre-curing zone of the tubular column, which has a temperature which is below the melting temperature of the matrix material. Preferably, the second continuous phase is introduced into the pre-curing zone via a second fluid inlet unit of the bottom portion of the curing device.
d. Delivering the dispersion and the second continuous phase together into a stirring zone of the tubular column. A radial stirring device is arranged inside the tubular column and extends along a longitudinal axis of the curing device from a head portion of the curing device only into the stirring zone of the tubular column. The stirring device radially mixes the dispersion and the second continuous phase with each other in the stirring zone.
e. Cooling the dispersion of droplets in the tubular column, in particular in the pre-curing zone and in the stirring zone, thereby solidifying the matrix material and generating cured capsules. Preferably, cooling is effected by means of the second continuous phase.

It is understood that the references a. to e. of the above mentioned steps do not indicate a specific order of the steps, but merely serves to identify and reference the steps. Thus it may well be possible that certain steps are performed together, or it may be possible that for example step c. commences before step b. or vice versa. Furthermore, the term “melting temperature” or “melting point” may not necessarily be a single specific temperature but may also refer to a melting temperature range, in particular if a mixture of different components is used as matrix material. In some embodiments, step d. is performed after steps b and c., i.e. the dispersion and the second continuous phase are first introduced into the pre-curing zone, flow through the pre-curing zone and are then delivered together into the stirring zone.

The method according to the third aspect may be performed with a curing device and/or a capsule production device according to any of the embodiments described herein.

In certain embodiments, the ratio of the viscosity of the molten matrix material to the viscosity of the first continuous phase may be between 0.05:1 and 5:1 in particular between 0.1:1 and 1:1.

As mentioned above, the continuous phase which is employed in the dispersion of droplets as the continuous phase is termed as “first continuous phase”. The continuous phase being in step c. introduced into the tubular column of the curing device via the second fluid inlet unit of the curing device may be termed as “second continuous phase”. The first and second continuous phase may be the same, e.g. both be water or an alcohol, such as polyvinyl alcohol, or may be different from each other. However, typically, the first and second continuous phase are miscible with each other at room temperature (20° C.) and normal pressure (1 atm.).

The temperature of the second continuous phase and/or the first continuous phase is typically selected such that a mixing temperature of the second continuous phase and the dispersion of droplets which comprise a molten matrix material, in a first continuous phase, is below the melting temperature of the matrix material. Furthermore, the temperature of the first continuous phase may preferably be selected such that the molten matrix material remains in a molten state when being contacted with only the first continuous phase.

In some embodiments, the droplets comprise in addition to the molten matrix material one or more compounds of interest. The compound of interest may for example be selected from a flavor, a fragrance, an enzyme, an active pharmaceutical ingredient, such as a medicament and the like.

In preferred embodiments, the method according to the embodiments of the third aspect is performed with a curing device according to any of the embodiments described herein, in particular with respect to the first aspect and/or with a capsule production unit according to any of the embodiments described herein, in particular with respect to the second aspect.

In some embodiments, prior to step a. a temperature equilibrium between the first continuous phase and the second continuous phase is established by purging the tubular column only with the first and second continuous phase, before step b. i.e. before the dispersion is introduced into the curing device. This increases the capsule quality and provides more uniform capsules.

In some embodiments, a rotational direction of the radial stirring device is switched between clockwise and counter clockwise rotation at predefined points in time. The rotation in the clockwise, respectively counter-clockwise, direction is conducted for a predefined time interval before it is switched to counter-clockwise, respectively clockwise direction for a predefined time interval. Each interval may have a duration of 1 s to 100 s, in particular of 5 s to 30 s. This allows to break a Taylor-Couette flow generating due to the radial stirring, which in turn avoid agglomeration of the capsules.

The second continuous phase has preferably a temperature of at least 5° C. below, in particular of at least 10° C., more particular of at least 12° C. below the melting temperature of the matrix material. In certain embodiments, the second continuous phase has a temperature of 5° C. to 20° C. below, in particular of 10° C. to 20° C. below, more particular of 12° C. to 15° C. below the melting temperature of the matrix material. Cooling at least below 10° C. of the melting temperature is beneficial as particular pure matrix material without small impurities which typically accelerate solidification tend to only slowly solidify if a higher temperature is selected.

In some embodiments, the method further comprises step f. Step f. comprises removing the generated and cured capsules from the tubular column and/or the curing device via a fluid outlet of the head portion of the curing device.

In some embodiments, step a. includes sub-steps a1) to a3). Sub-step a1) comprises melting the matrix material in a melting unit to provide a molten matrix material. Sub-step a2) comprises transporting the molten matrix material into the emulsification device. Sub-step a3) comprises mixing the molten matrix material with the first continuous phase such that the dispersion of droplets comprising a molten matrix material in the first phase is provided. Sub-step a3) may preferably be conducted in an emulsification device, in particular an emulsification device as described in the first aspect of the invention.

Typically, the temperature of the first continuous phase may generally be selected such that it has a temperature at or above the melting temperature of the matrix material.

In certain embodiments, sub-step a2) is performed by a dosing unit, e.g. a dosing unit as described in any of the embodiments herein. The dosing unit may withdraw the molten matrix material from the melting unit and particularly deliver it into the dosing unit, by the application of a negative pressure. Thereafter, the dosing unit delivers the molten matrix material from the dosing unit to the emulsification device by application of a positive pressure.

In some embodiments, withdrawing the molten matrix material from the melting unit and providing the molten matrix material from the dosing unit to the emulsification device is controlled by a valve being arranged between and fluidic connected to the dosing unit, the melting unit and the emulsification device. The valve provides, during withdrawing of the molten matrix material from the melting unit, an exclusive fluidic connection between the melting unit and the dosing unit. Thereby, the molten matrix material is guided into the dosing unit. Then the valve is switched such that it provides an exclusive fluidic connection between the dosing unit and the emulsification device. Then the dosing unit applies a positive pressure and delivers the molten matrix material, in particular the molten oil phase, from the dosing unit to the emulsification device.

A fourth aspect of the invention relates to a processing unit as such as described with respect to any of the embodiments of the second aspect. As outlined above, such a processing unit comprises a melting unit and a dosing unit. The melting unit defines, respectively comprises, a melting compartment. Furthermore, the melting unit comprises a heating unit which is configured for heating a matrix material within the melting compartment. The dosing unit is configured for providing a molten matrix material, in particular a molten oil phase from the melting unit to an emulsification device. The melting unit, the dosing unit and/or the optional valve can comprise any of the features as described herein, in particular with respect to the second and third aspect of the invention.

### BRIEF DESCRIPTION OF THE FIGURES

The herein described invention will be more fully understood from the detailed description given herein below and the accompanying drawings which should not be considered limiting to the invention described in the appended claims. The drawings are showing:

FIG. 1 a front view of a curing device according to an embodiment of the invention;

FIG. 2 a sectional view of the embodiment shown in FIG. 1 along A-A;

FIG. 3 an exploded view of a curing device according to another embodiment of the invention;

FIG. 4 a view from the perspective of the tubular column onto a bottom portion of a curing device according to an embodiment of the invention;

FIG. 5 a sectional view of the bottom portion of FIG. 4 along B-B;

FIG. 6 a perspective view of the bottom portion of FIG. 4;

FIG. 7 a perspective view of a capsule production unit according to an embodiment of the invention;

FIG. 8 a perspective view of a capsule production unit according to another embodiment of the invention;

FIG. 9 a front view of a melting unit as it can be used in some embodiments of a capsule production unit according to some embodiments of the invention;

FIG. 10 a sectional view of the melting unit of FIG. 9 along C-C;

FIG. 11 a schematic representation of a capsule production unit according to another embodiment of the invention.

### EXEMPLARY EMBODIMENTS

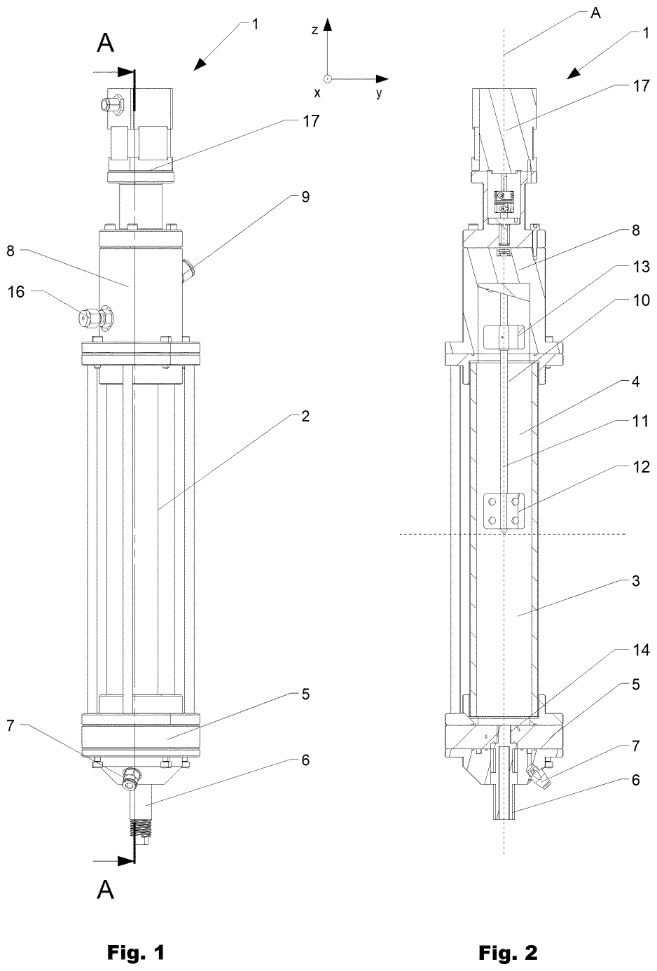

FIG. 1 and FIG. 2 show a curing device 1 according to an embodiment of the invention. Curing device 1 comprises bottom portion 5 and head portion 8 as well as tubular column 2 being arranged and sandwiched between bottom portion 5 and head portion 8. In this embodiment, the curing device comprises further a plurality of longitudinally extending rods, which extend in parallel to tubular column 2 and may primarily serve a stabilizing and protecting function, but are not essential for the device. Tubular column 2 has a longitudinal axis A which is illustrated in FIG. 2 by the dashed vertical line. Longitudinal axis A extends along an axial direction z of tubular column 2. The horizontal plane, which may be referred to as the radial plane is arranged perpendicular to longitudinal axis A and axial direction z and is thus in the x-y plane. Tubular column 2 defines two zones, namely pre-curing zone 3 and stirring zone 4. The pre-curing zone 3 extends between bottom portion 5 and stirring zone 4, i.e. as shown in FIG. 2 up to radial stirring device 10. Stirring zone 4 is arranged adjacent to pre-curing zone 3 and extends between pre-curing zone 3 and head portion 8. In FIG. 2, pre-curing zone 3 and stirring zone 4 are separated from each other by the horizontal dashed line. Curing device 1 further comprises radial stirring device 10, which is arranged inside tubular column 2 and particularly inside stirring zone 4, but generally not in pre-curing zone 3. Radial stirring device 10 extends along longitudinal axis A from head portion 8 only into stirring zone 4 and thus not into pre-curing zone 3. Furthermore, radial stirring device 10 is configured for providing a radial mixing, i.e. a mixing in the x-y-plane, of a dispersed phase in a continuous phase within tubular column 2. Radial stirring device 10 includes two stirring elements 12 and 13 which each extend radially away from stirring rod 10.

Bottom portion 5 comprises first fluid inlet unit 6 which is used for introducing the dispersed phase of droplets in a first continuous phase into tubular column 2 and in particular into pre-curing zone 3. First fluid inlet unit 6 comprises inlet tube 14 being centrally arranged and/or being coaxially arranged with longitudinal axis A. Additionally, bottom portion 5 comprises second fluid inlet unit 7 being different from first fluid inlet unit 6 and which is used for introducing a second continuous phase into tubular column 2, in particular into pre-curing zone 3. Head portion 8 comprises fluid outlet 9 which is in this embodiment angled with respect to longitudinal axis A in angle (i.e. generally the acute angle between longitudinal axis A and fluid outlet 8 opening away from the bottom portion 5) of between 10° to 70°. Head portion 8 further comprises in this embodiment drive unit 17 which is configured to drive rotation of radial stirring device 10. Head portion 8 further comprises temperature sensor unit 16 which is configured for measuring the temperature of the continuous phase, i.e. the first and second continuous phase, within curing device 1, in particular within tubular column 2.

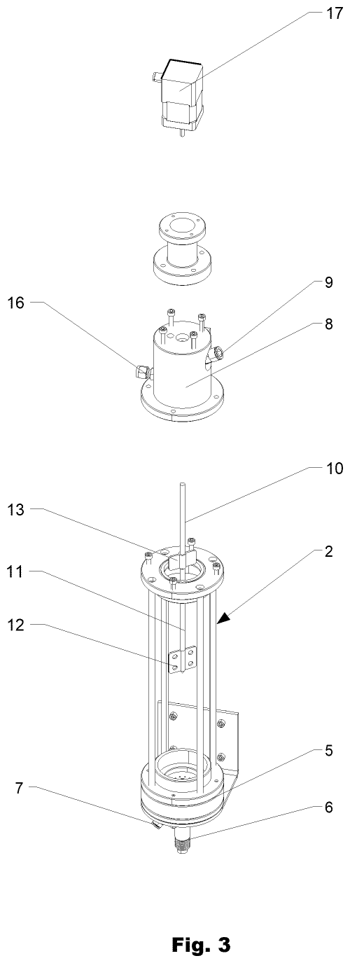

FIG. 3 shows an exploded view of curing device 1 according to another embodiment of the invention. For reasons of clarity, the column wall of tubular column 2 has been removed to show the interior of curing device 1.

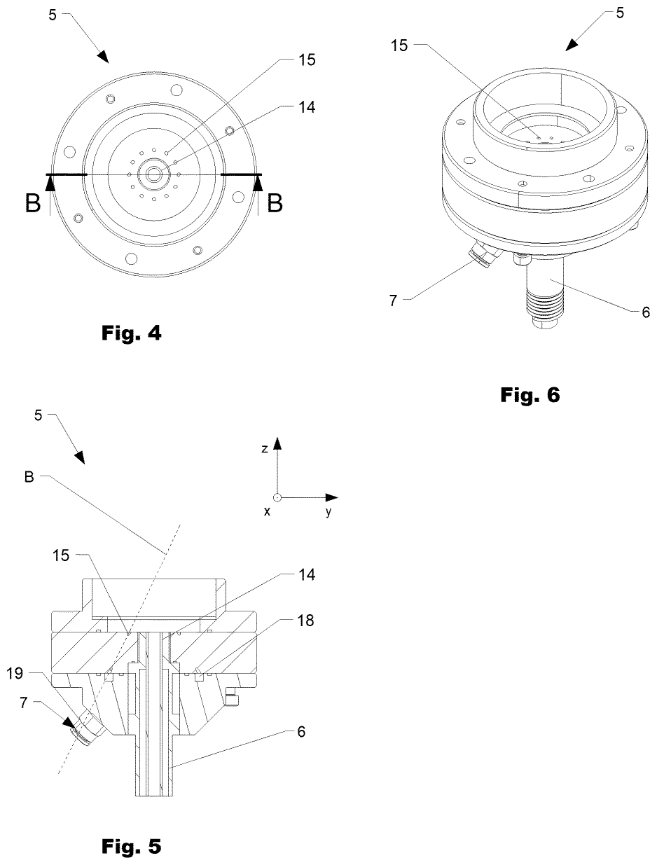

FIG. 4 to 6 show bottom portion 5 as it can be employed in a curing device according to the invention and serve to illustrate further details on first fluid inlet unit 6 and second fluid inlet unit 7. As can be seen, second fluid inlet unit 7 comprises a plurality of second inlets 15 (for clarity purposes only a single second inlet is referenced) which surround first inlet tube 14 of first inlet unit 6. As can be seen, second inlets 15 are arranged on a circle surrounding first inlet tube 14, which allows to directly and efficiently bring the droplets in contact with the second continuous phase to initiate curing in the pre-curing zone. As can be seen from the sectional view of FIG. 5, each second inlet 15 can define an inlet axis B, along which the second continuous phase is provided into the second chamber and/or along which the second inlet extends at least partially or fully. Inlet axis B is angled in an angle of below 90° towards the horizontal plane, i.e. the radial plane in the x-y plane being perpendicular to axial direction z and thus to the longitudinal axis of the curing device. Furthermore, the plurality of second inlets 15 are arranged and configured such that they generate a vortex of the second continuous phase when it is introduced via the plurality of second inlets into the pre-curing zone of the tubular column. This vortex extends around longitudinal axis A, which may be the center of the vortex. Furthermore, second fluid inlet unit 7 comprises flow channel 18 and second inlet tube 19. Flow channel 18 circumferentially surrounds first inlet tube 14 and provides a fluid connection between second inlet tube 19 and each of the plurality of second inlets 15.

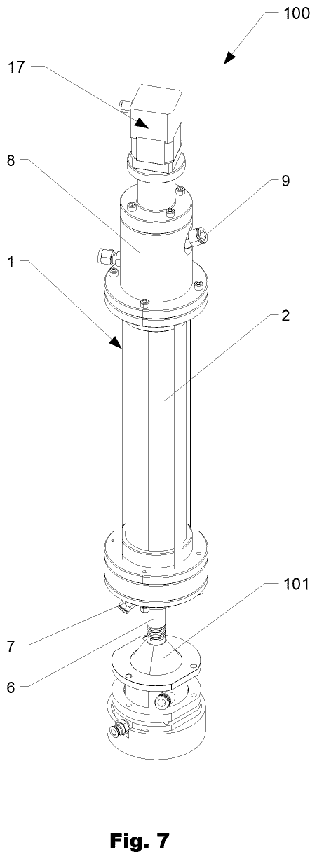

FIG. 7 shows a capsule production unit 100 according to an embodiment of the invention. Capsule production unit 100 comprises curing device 1, such as curing device 1 shown in FIG. 1 to 3. Further, emulsification unit 100 comprises emulsification device 101, which is connected to first fluid inlet unit 6 of curing device 1. Emulsification device 101 can produce a dispersion of droplets in a first continuous phase, by introducing a molten matrix material forming the droplets into a first chamber of the emulsification device, transporting the molten matrix material through one or more channels into a second chamber of the emulsification device comprising a first continuous phase. The first continuous phase is typically continuously delivered into the second chamber and the molten matrix material is typically continuously delivered into the first chamber. Thereby, the continuously produced dispersion of droplets in the first continuous phase is delivered directly via first fluid inlet unit 6 of curing device 1 into the tubular column 2 of curing device 1 and particularly first into the pre-curing zone. Concomitantly, a second continuous phase is delivered into curing device 1 and tubular column 2 via second fluid inlet unit 7. The second continuous phase has a temperature which is lower than the melting temperature of the matrix material of the droplets, for example around 10 to 15° C. lower. Thereby, the droplets commence curing in the pre-curing zone and reach a sufficient curing level before they enter the stirring zone of tubular column 2. Thereby, capsule agglomeration, quality decrease and damage is avoided.

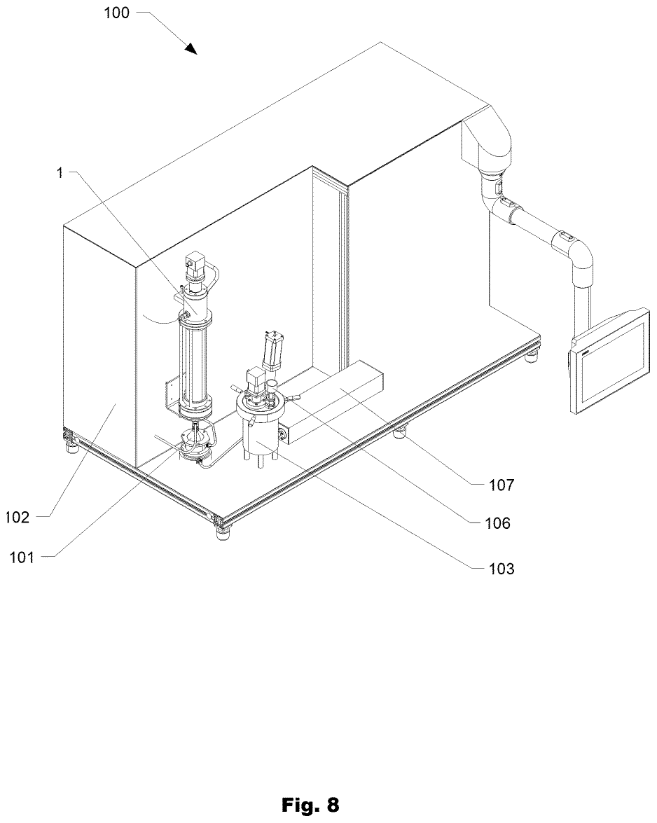

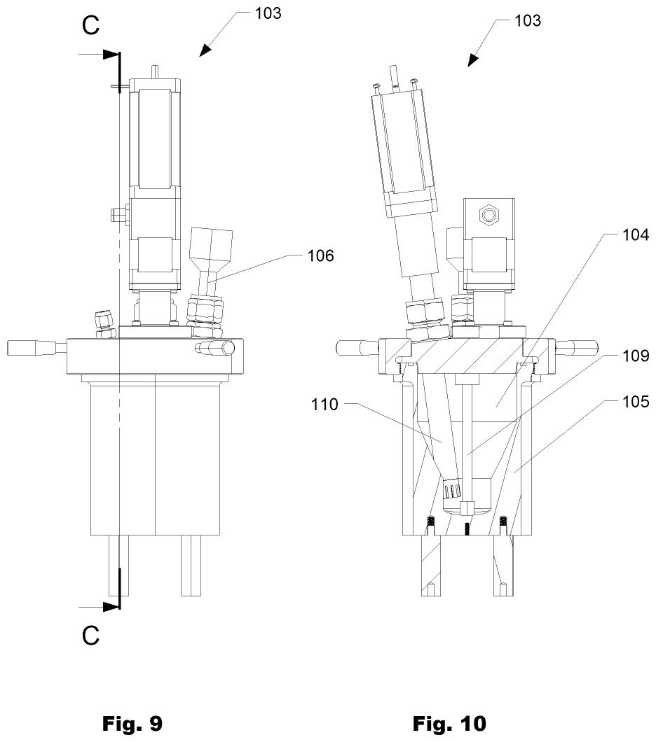

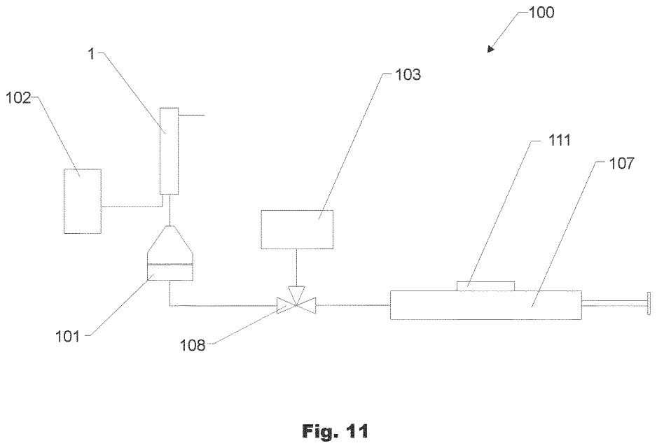

FIG. 8 shows a capsule production unit 100 according to another embodiment of the invention. In this embodiment, capsule production unit 100 not only comprises emulsification device 101 as the capsule production unit shown in FIG. 7, but additionally comprises a processing unit. The processing unit includes dosing unit 107 and melting unit 103 which is shown in further detail in FIGS. 9 and 10. Melting unit 103 is in fluidic connection with dosing unit 107 and directly or alternatively, indirectly via dosing unit 107, also with emulsification device 101. Capsule production unit 100 further comprises a continuous phase reservoir 102 which may be used to supply the first continuous phase to the emulsification device 101 and/or the second continuous phase to the second fluid inlet unit of curing device 1.

As can be seen in more detail in FIGS. 9 and 10, melting unit 103 defines melting compartment 104 and heating unit 105 which is configured to heat any material, such as a matrix material in melting compartment 104. For readily delivering solid or viscous matrix material into melting compartment 104, melting unit 103 further comprises matrix material inlet 106 having a funnel shape. Melting unit 103 further comprises stirrer 109, which in this embodiment is for example an anchor stirrer. Stirrer 109 is driven by a corresponding drive unit. Melting unit 103 additionally comprises emulsifier unit 110, which is partially arranged inside melting compartment 104. Emulsifier unit 110 also includes a drive unit, which drives the emulsifier. When a hydrophilic compound of interest or a hydrophilic phase shall be present in the molten matrix material, the emulsifying unit may enable the formation of such an emulsion of the hydrophilic compound of interest or hydrophilic phase in the generally lipophilic matrix material. Furthermore, the emulsifying unit may generally be configured for providing a suspension of a solid compound of interest and the matrix material. This may be beneficial if a sloid is envisioned to be included into the capsules. Additionally, when a solid matrix material, such as a wax, is introduced into melting compartment 104, emulsifier unit may comminute and/or grind the matrix material and therefore allows to rapidly achieve a homogenous phase. Furthermore, emulsifier unit 110 may help to comminute and/or mill compounds of interest which shall be contained in the capsules to be produced. In such embodiments, the compound of interest may also be introduced into the melting compartment together with the matrix material. Then, the mixture is heated beyond the melting temperature of the matrix material to form a liquid dispersed phase.

FIG. 11 shows a schematic representation of capsule production unit 100. In addition to melting unit 103 and dosing unit 107, the processing unit also includes valve 108. As can be seen, valve 108 is arranged between and connected to emulsification device 101, melting unit 104 and dosing unit 107. In this embodiment, dosing unit 107 comprises a syringe pump. In a typical process, matrix material is molten and optionally mixed with a compound of interest in melting unit 103 to provide a molten matrix material which has a lower viscosity than the solid (i.e. initial) matrix material and is preferably liquid. The valve is in a position in which it exclusively connects melting unit 103 with dosing unit 107. Then, dosing unit 107 applies a negative pressure and withdraws the molten matrix material from the melting compartment of melting unit 103. Thereby, the molten matrix material is received in the dosing unit. A dosing unit heater 111 ensures that the matrix material does not solidify inside dosing unit 107. As a next step, valve 108 is switched to a position in which it exclusively connects dosing unit 107 with emulsification device 101. The dosing unit then applies a positive pressure and forces the molten matrix material out of the dosing unit and via valve 108 into emulsification device 101. Therein, the molten matrix material serves as the dispersed phase and is forced through one or more channels in a membrane separating a first chamber of emulsification device 101 from a second chamber. A first continuous phase being immiscible with the molten matrix material is introduced into the second chamber and droplets of the matrix material form. The thus produced dispersion of droplets of the molten matrix material in the first continuous phase is then delivered via the corresponding first fluid inlet unit of curing device 1 into the tubular column of the curing device, where it is contacted with a second continuous phase, which may be the same or different then the first continuous phase. Since the second continuous phase has a temperature below the melting temperature of the matrix material, curing is effected and capsules are generated. The second continuous phase may be delivered into the tubular column of curing device 1 via second fluid inlet unit of the curing device from continuous phase reservoir 102.

### LIST OF DESIGNATIONS

1 curing device
2 tubular column
3 pre-curing zone
4 stirring zone
5 bottom portion
6 first fluid inlet unit
7 second fluid inlet unit
8 head portion
9 fluid outlet
10 radial stirring device
11 stirrer rod
12, 13 stirring element
14 first inlet tube
15 second inlet
16 temperature sensor unit
17 drive unit
18 flow channel
100 capsule production unit
101 emulsification device
102 continuous phase reservoir
103 melting unit
104 melting compartment
105 heating unit
106 matrix material inlet
107 dosing unit
108 valve
109 stirrer
110 emulsifier unit
111 dosing unit heater
A longitudinal axis
B inlet axis

1. A curing device for generating and curing capsules the curing device comprising:
a. a tubular column having a longitudinal axis, which extends along an axial direction of the tubular column, wherein the tubular column defines along the longitudinal axis a pre-curing zone and an adjacent stirring zone;
b. a bottom portion comprising a first fluid inlet unit for introducing a dispersed phase into the tubular column and a second fluid inlet unit for introducing a continuous phase into the tubular column, wherein the pre-curing zone extends between the bottom portion and the stirring zone;
c. a head portion comprising a fluid outlet for removing the generated and cured capsules from the curing device;
d. a radial stirring device being arranged inside the tubular column-(and inside the stirring zone of the tubular column, wherein the radial stirring device is configured to provide a radial mixing of the dispersed phase and the continuous phase within the tubular column, wherein the radial stirring device extends along the longitudinal axis from the head portion only into the stirring zone.

2. The curing device according to claim 1, wherein the radial stirring device comprises a stirrer rod and one or more stirring elements being connected to the stirrer rod and extending at least partially radially from the stirrer rod.

3. The curing device according to claim 1, wherein the first fluid inlet unit comprises a first inlet tube and wherein the second fluid inlet unit comprises a plurality of second inlets surrounding the first inlet tube.

4. The curing device according to claim 3, wherein the plurality of second inlets is configured such that they can generate a vortex of the continuous phase upon introduction into the tubular column.

5. The curing device according to claim 3, wherein the plurality of second inlets is arranged radially around the first inlet tube.

6. The curing device according to claim 3, wherein each second inlet defines an inlet axis along which it extends and wherein each inlet axis is angled towards a horizontal plane which is perpendicular to the longitudinal axis in an angle of below 90°.

7. The curing device according to claim 3, wherein the first inlet tube comprises a heating element, such as a heating coil, being configured to provide thermal energy to the dispersed phase flowing therethrough.

8. The curing device according to claim 1, wherein a length of the pre-curing zone is at least 20% of a total length of the tubular column.

9. (canceled)

10. The curing device according to claim 1, wherein the curing device comprises a temperature sensor unit being configured for measuring the temperature of the continuous phase inside the tubular column.

11. The curing device according to claim 1, wherein the fluid outlet of the head portion defines an outlet axis being angled to the longitudinal axis in an angle of 10° to 90°.

12. (canceled)

13. A capsule production unit comprising:
a. a curing device according to claim 1; and
b. an emulsification device being configured for generating a dispersion of droplets in a continuous phase and being in fluidic communication with the first fluid inlet unit of the curing device;
c. optionally a continuous phase reservoir being in fluidic communication with the second fluid inlet unit of the curing device.

14. The capsule production unit according to claim 13, further comprising a processing unit, the processing unit comprising:
a. a melting unit defining a melting compartment, wherein the melting unit comprises a heating unit and optionally a matrix material inlet for delivering a matrix material into the melting compartment;
b. a dosing unit being configured for providing a molten matrix material from the melting unit to the emulsification device.

15. The capsule production unit according to claim 14, wherein the processing unit further comprises a valve being fluidically connected to the melting unit, the dosing unit and the emulsification device and providing a fluidic connection between the melting unit, the dosing unit and the emulsification device.

16. The capsule production unit according to claim 15, wherein the dosing unit is configured for establishing a negative pressure to transport the molten matrix material from the melting unit into the dosing unit via the valve and/or for establishing a positive pressure to transport the molten matrix material from the dosing unit to the emulsification device via the valve.

17. The capsule production unit according to claim 14, wherein the melting unit further comprises one or more of a stirrer and an emulsifier unit.

18. (canceled)

19. The capsule production unit according to claim 14, wherein the dosing unit comprises a dosing unit heater being configured for heating the dosing unit and/or the molten matrix material inside the dosing unit.

20. A method for generating and curing capsules, the method comprising the steps:
a. providing a dispersion of droplets comprising a molten matrix material in a first continuous phase;
b. introducing the dispersion into a pre-curing zone of a tubular column of a curing device;
c. introducing a second continuous phase into the pre-curing zone of the tubular column 2 wherein the second continuous phase has a temperature below the melting temperature of the matrix material;
d. delivering the dispersion and the second continuous phase into a stirring zone of the tubular column, wherein a radial stirring device is arranged inside the tubular column and extends along a longitudinal axis of the curing device from a head portion of the curing device only into the stirring zone of the tubular column, wherein the radial stirring device radially mixes the dispersion and the second continuous phase in the stirring zone;
e. cooling the dispersion of droplets in the tubular column in the pre-curing zone and the stirring zone thereby solidifying the matrix material and generating cured capsules.

21. (canceled)

22. The method according to claim 20, wherein step a. includes steps: a1) melting a matrix material in a melting unit to provide a molten matrix material; a2) transporting the molten matrix material into an emulsification device; a3) mixing the molten matrix material with the first continuous phase such that the dispersion of droplets comprising a molten matrix material in the first continuous phase is provided.

23. The method according to claim 22, wherein step a2) is performed by a dosing unit, wherein the dosing unit withdraws the molten matrix material from the melting unit by application of a negative pressure and wherein thereafter the dosing unit delivers the molten matrix material from the dosing unit to the emulsification device by application of a positive pressure.

24. The method according to claim 23, wherein withdrawing the molten matrix material from the melting unit and providing the molten matrix material from the dosing unit to the emulsification device is controlled by a valve being arranged between and fluidically connected to the dosing unit, the melting unit and the emulsification device.
